(19) United States
(12) Patent Application Publication
(10) Pub. No.: US 2005/0080072 A1
(43) Pub. Date: Apr. 14, 2005

(54) Process for the preparation of a thiazepine derivative

(75) Inventors: Pandurang Balwant Deshpande, Chennai (IN); Anil Ganpat Holkar, Chennai (IN); Omprakash Gudaparthi, Chennai (IN); Jothi Dinesh Kumar, Cuddalore (IN)

Correspondence Address: OLIFF & BERRIDGE, PLC, P.O. BOX 19928, ALEXANDRIA, VA, 22320, US

(73) Assignee: ORCHID CHEMICALS & PHARMACEUTICALS LTD., Chennai (IN)

(21) Appl. No.: 10/925,941

(22) Filed: Aug. 26, 2004

Related U.S. Application Data
(60) Provisional application No. 60/534,100, filed on Jan. 5, 2004.

(30) Foreign Application Priority Data
Sep. 1, 2003 (IN) 695/CHE/2003

(51) Int. Cl. A61K 31/554; C07D 417/04

(52) U.S. Cl. 514/211.13; 540/547

(57) ABSTRACT

The present invention relates to a process for the preparation of biologically active thiazepine derivative. The present invention more particularly, relates to an improved process for the preparation of dibenzo[b,f][1,4]thiazepine derivative of formula (I).

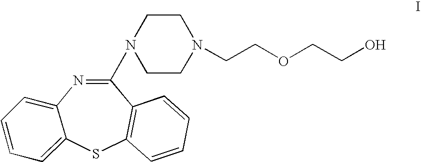

### FIELD OF THE INVENTION

The present invention relates to a process for the preparation of biologically active thiazepine derivative. The present invention more particularly, relates to an improved process for the preparation of dibenzo[b,f][1,4]thiazepine derivative of formula (I).

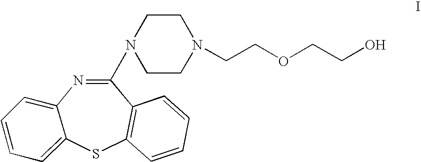

### DESCRIPTION OF THE PRIOR ART

Biologically important compound of dibenzo[b,f][1,4]thiazepine derivative is 11-[4-[2-(2-hydroxyethoxy)ethyl]-1-piperazinyl]dibenzo[b,f][1,4]thiazepine generically known, as quetiapine. Quetiapine fumarate is an antipsychotic agent useful for the treatment of schizophrenia and related disease conditions by antiagonising dopamine and/or serotonin receptor.

U.S. Pat. No. 4,879,288 originally disclosed Quetiapine and its salts. The patent also discloses various processes for the preparation of quetiapine which are given below: 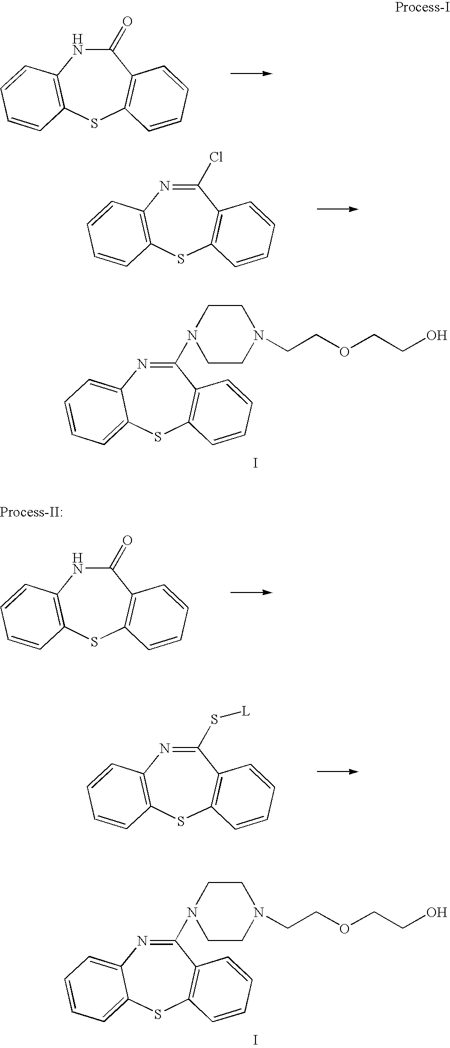
wherein S-L represents a leaving group.

WO 01/55125 discloses a process for the preparation of quetiapine which are given below:

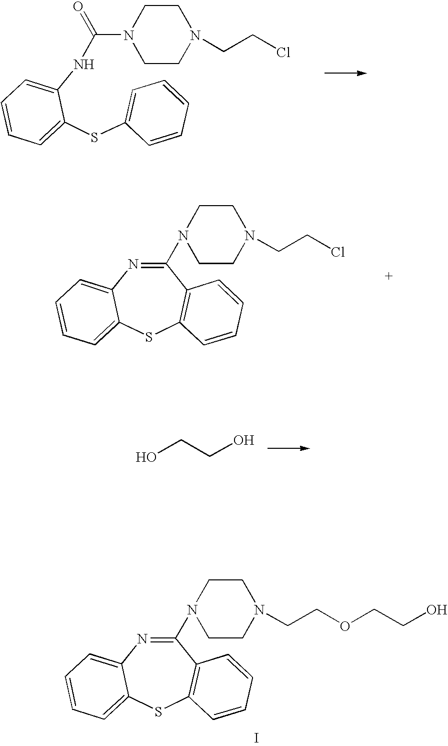

EP 0 282 236 discloses a process which is given below: 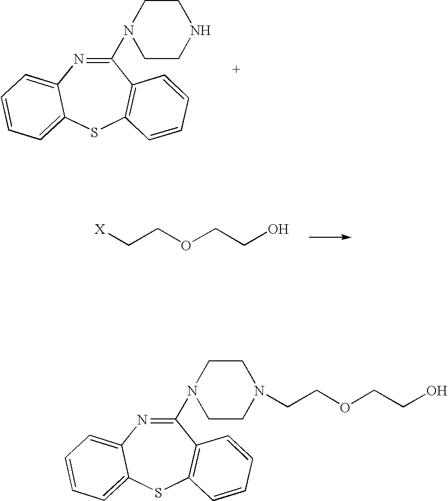
wherein X is a leaving group.

In view of the biological importance of quetiapine and to have a commercially viable, easily scalable process, we focused our research to develop an improved process for the preparation of the compound of formula (I), which process has advantages over the processes described in the above-mentioned prior art documents.

### OBJECTIVES OF THE INVENTION

The main objective of the present invention is to provide an improved process for the preparation of compound of general formula (I).

Another objective of the present invention is to provide a process for the preparation of compound of formula (I), which would be easy to implement on commercial scale.

Still another objective of the present invention is to provide a process for the preparation of compound of formula (I) in good yield and high purity.

Yet another objective of the present invention is to provide novel intermediates of formula (VI), (VII), (VIII) and (IX).

### SUMMARY OF THE INVENTION

Accordingly, the present invention provides an improved process for the preparation of compound of formula (I) 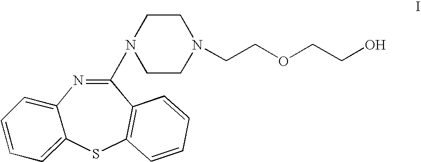
the said process comprising the steps of:
(i) reacting the compound of formula (II) wherein X represents a leaving group, with compound of formula (III) in presence of base and a solvent to produce acid of formula (IV),
(ii) converting the acid of formula (IV) into acid derivative of formula (V) wherein R is a group which forms a basis that a compound of formula (V) in a reactive form including halogen atoms selected from chlorine ,bromine; a group which forms together with the —C═O group to which R is attached an active ester and a group which forms together with the —C═O group to which Y is attached a mixed anhydride in the presence of a base and solvent to produce the compound of formula (V),
(iii) reacting the compound of formula (V) with compound of formula (X) in the absence or presence of base and the absence or presence of a solvent to produce a compound of formula (VI),
(iv) converting the compound of formula (VI) into compound of formula (VII), wherein R₁ represents an alcohol protecting group, using a reagent, in the presence or absence of a base and solvent,
(v) reducing the compound of formula (VII) using a reducing agent in the presence of a solvent to produce compound of formula (VIII),
(vi) cyclizing the compound of formula (VIII) using cyclising agent in the presence of a solvent to produce compound of formula (IX), and
(vii) deprotecting the compound of formula (IX) using basic reagent in the presence of solvent to produce a compound of formula (I).

The process is shown in scheme given below:

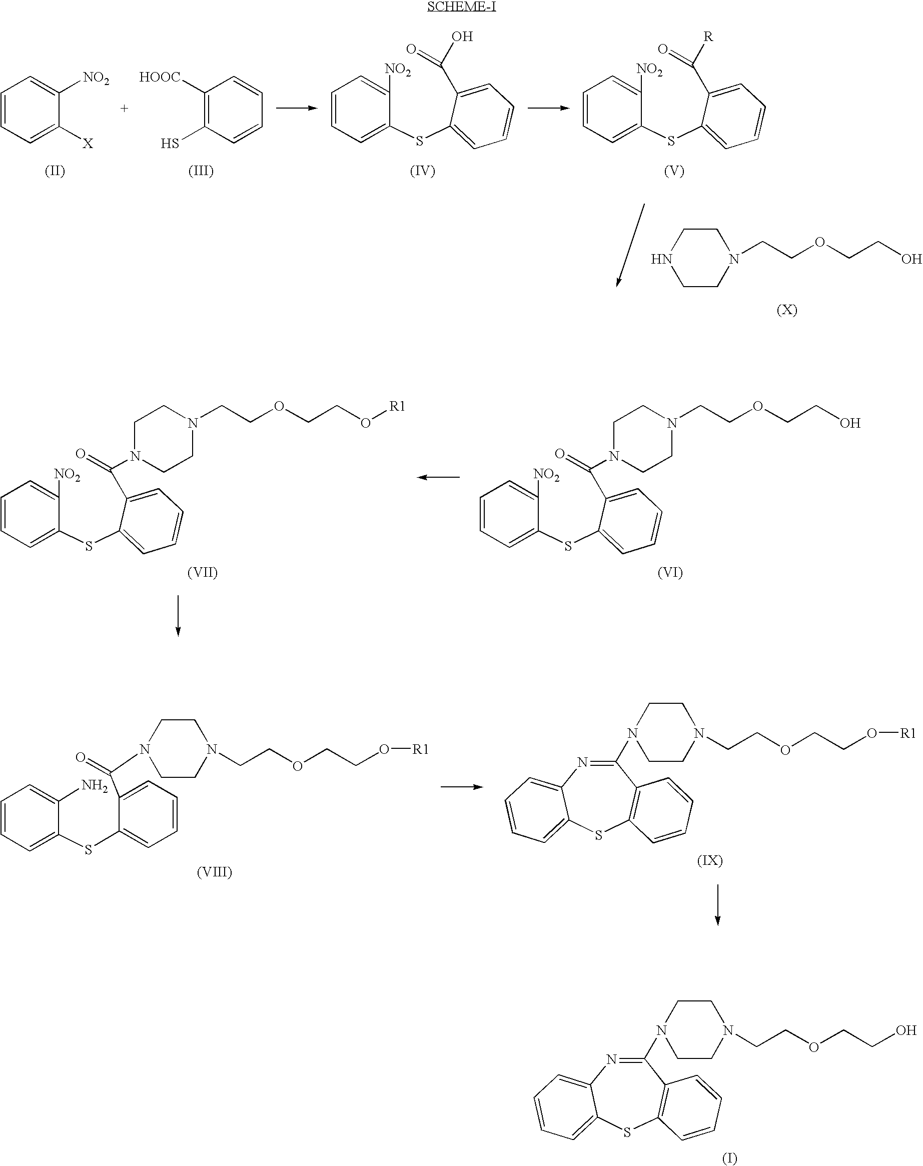

In still another embodiment of the present invention there is provided a novel intermediate of formula (VI), used in the production of Quetiapine.

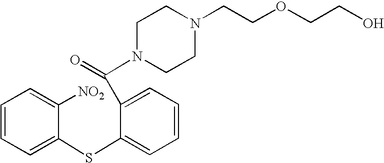

In yet another embodiment of the present invention there is provided a novel intermediate of formula (VII) used in the production of Quetiapine. 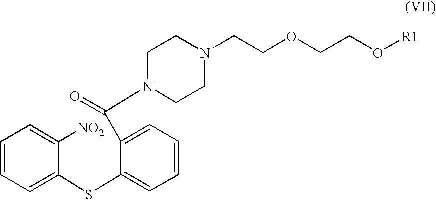
where R₁ is alcohol protecting group such as acyl, alkyl, trityl, or benzyl.

In yet another embodiment of the present invention there is provided a novel intermediate of formula (VIII), used in the production of Quetiapine.

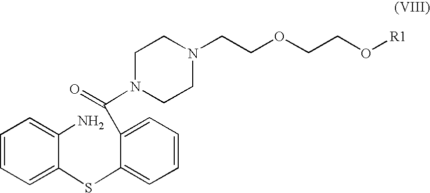

In another embodiment of the present invention there is provided a novel intermediate of formula (IX), used in the production of Quetiapine. 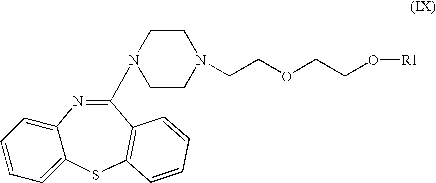
where R₁ is alcohol protecting group such as acyl, alkyl, trityl, or benzyl.

### DETAILED DESCRIPTION OF THE INVENTION

In an embodiment of the present invention the leaving group represented by X is selected from halogen such as fluoro, chloro, bromo, iodo.

In another embodiment of the present invention the solvent employed in step (i) is selected from esters such as ethyl acetate, methyl acetate; ethers such as IPE, diethyl ether; alkanols such as methanol, ethanol, IPA; nitrile such as acetonitrile; ketones such as acetone, ethyl methyl ketone; hydrocarbon such as benzene, toluene, hexane; halogenated hydrocarbon such as dichloro methane, dichloro ethane; Dimethyl formamide (DMF), Dimethyl Acetamide (DMAc), diglyme, N-methylpyrrolidone, hexamethyl phosphoramide (HMPA), monoglyme, THF, dioxane, dimethylsulphoxide (DMSO), sulpholane, and the like or mixtures thereof.

In still another embodiment of the present invention the base employed in step (i) is selected from alkali/alkaline earth metal hydroxide such as sodium hydroxide, potassium hydroxide; alkali/alkaline earth metal carbonates such as sodium carbonate, potassium carbonate; alkali/alkaline earth metal bicarbonates such as potassium bicarbonate, metal alkoxide such as sodium methoxide, potassium tert-butoxide; metal amides, metal hydrides such as sodium hydride; organic amines such as triethyl amines, N-methyl-morpholine, diethyl amine, pyridine, DBU, DBN and the like.

In yet another embodiment of the present invention the reagent employed in step (ii) is selected from, SOCl₂, PCl₅, PCl₃, POCl₃, pivaloyl chloride, methyl chloro formate ethyl chloroformate, 2,4,6-trichlorobenzoyl chloride, N,N′-carbonyldiimidazole, diethylchlorophophite, diphenylphosphorochloridate, 1-hydroxybenzotriazole and the like or mixtures thereof.

In yet another embodiment of the present invention the organic solvent employed in step (ii) is selected from esters such as ethyl acetate, methyl acetate; ethers such as IPE, diethyl ether; alkanols such as methanol, ethanol, IPA; nitrile such as acetonitrile; ketones such as acetone, ethyl methyl ketone; methyl isobutyl ketone hydrocarbon such as benzene, toluene, hexane; halogenated hydrocarbon such as dichloro methane, dichloro ethane; amides such as DMF, DMAc, N-methylpyrrolidone, hexamethyl phosphoramide (HMPA), diglyme, monoglyme, THF, dioxane, and the like or mixtures thereof; and the base employed in step (ii) is selected from organic amines such as triethyl amine, N-methyl-morpholine, diethyl amine, pyridine or mixtures thereof.

In another embodiment of the present invention the acid derivative group represented by R is selected from chloro, bromo, iodo, ethoxy carbonyl, trimethyl acetyl, 2,4,6-trichlorobenzyl, or acid activating agents such as carboimides, N,N′-carbonyldiimidazole, and the like.

In another embodiment of the present invention the step (ii) and step (iii) can be carried out in situ manner in a single pot.

In still another embodiment of the present invention, the base employed in step (iii) is selected from organic amines, alkali/alkaline earth metal carbonates, alkali/alkaline earth metal bicarbonates and the solvent employed is selected from esters such as ethyl acetate, methyl acetate; ethers such as IPE, diethyl ether; alkanols such as methanol, ethanol, IPA; nitrile such as acetonitrile; ketones such as acetone, ethyl methyl ketone; methylisobutyl ketone, hydrocarbon such as benzene, toluene, hexane; halogenated hydrocarbon such as dichloro methane, dichloro ethane; amides such as DMF, DMAc, N-methylpyrrolidone, hexamethyl phosphoramide (HMPA); diglyme, monoglyme, THF, dioxane, and the like or mixtures thereof.

In another embodiment of the present invention the alcohol protecting group represented by R₁ is acyl, alkyl, trityl, benzyl and the like.

In still another embodiment of the present invention, the base employed in step (iv) is selected from triethyl amine, N-methyl-morpholine, diethyl amine, pyridine and the like; and the reagent employed is selected from acetic anhydride, alkyl halide, dialkyl sulphate, trityl chloride, benzyl halide and the like.

In another embodiment of the present invention, the solvent employed in step (iv) is selected from esters such as ethyl acetate, methyl acetate; ethers such as IPE, diethyl ether; alkanols such as methanol, ethanol, IPA; nitrile such as acetonitrile; ketones such as acetone, ethyl methyl ketone; hydrocarbon such as benzene, toluene, hexane; halogenated hydrocarbon such as dichloro methane, dichloro ethane; amides such as DMF, DMAc, N-methylpyrrolidone, hexamethyl phosphoramide (HMPA), diglyme, monoglyme, THF, dioxane, inorganic acid, water and the like or mixtures thereof.

In yet another embodiment of the present invention, the reduction in step (v) is carried out using reducing agent selected from metals such as Zn, Sn or Fe/acid, AlH₃—AlCl₃, hydrazine hydrate ,/Raney nickel, sulfides such as NaHS, (NH₄)₂S, NaBH₂S₃, or by catalytic hydrogenation using Pd—C, Pt, Raney nickel and the solvent employed is selected from esters such as ethyl acetate, methyl acetate; ethers such as IPE, diethyl ether; alkanols such as methanol, ethanol, IPA; nitrile such as acetonitrile; ketones such as acetone, ethyl methyl ketone; hydrocarbon such as benzene, toluene, hexane; halogenated hydrocarbon such as dichloro methane, dichloro ethane; amides such as DMF, DMAc, N-methylpyrrolidone, hexamethyl phosphoramide (HMPA); diglyme, monoglyme, THF, dioxane, and the like or mixtures thereof; dimethylsulphoxide (DMSO), sulpholane, water, formic acid, acetic acid, ammonium formiate and the like or mixtures thereof.

In another embodiment of the present invention the cyclization in step (vi) is carried out either in acidic condition or basic condition.

In another embodiment of the present invention, the cyclization in step (vi) is carried out using acidic reagent such as polyphophosporic acid, methane sulphonic acid, PTSA, H₂SO₄, P₂O₅, P₂O₃, PCl₅, PCl₃, PBr₃, PBr₅, SOCl₂, superacids, anhydrides of superacids, Lewis acid, acetic acid, SO₂Cl₂, POCl₃, POBr₃, SOBr₂, formic acid, acetic anhydride, TiCl4, triflouro acetic acid, Al(CH₃)₃, acidic resins and the like. The reaction may be carried out in the presence of catalytic amounts of aniline, dialkyl amines, pyridine and the like.

In yet another embodiment of the present invention, the solvent employed in step (vi) is selected from esters such as ethyl acetate, methyl acetate; ethers such as IPE, diethyl ether; alkanols such as methanol, ethanol, IPA; nitrile such as acetonitrile; ketones such as acetone, ethyl methyl ketone; hydrocarbon such as benzene, toluene, hexane; halogenated hydrocarbon such as dichloro methane, dichloro ethane; DMF, DMAc, diglyme, monoglyme, THF, dioxane, dimethylsulphoxide (DMSO), sulpholane, water and the like or mixtures thereof

In another embodiment of the present invention the step (vi) and step (vii) can be carried out in situ manner in a single pot.

In another embodiment of the present invention the solvent employed in step (vii) is selected from esters such as ethyl acetate, methyl acetate; ethers such as IPE, diethyl ether; alkanols such as methanol, ethanol, IPA; nitrile such as acetonitrile; ketones such as acetone, ethyl methyl ketone; hydrocarbon such as benzene, toluene, hexane; halogenated hydrocarbon such as dichloro methane, dichloro ethane; DMF, DMAc, diglyme, monoglyme, THF, dioxane, dimethylsulphoxide (DMSO), sulpholane, water and the like or mixtures thereof.

In yet another embodiment of the present invention the basic reagent employed in step (vii) is selected from alkali/alkaline earth metal hydroxide such as sodium hydroxide, alkali/alkaline earth metal carbonates such as potassium carbonate, alkali/alkaline earth metal bicarbonates sodium bicarbonate, alkali/alkali earth metal oxides, metal alkoxide such as sodium methoxide, potassium tert-butoxide, metal amides, metal hydrides, organic amines, DBU, or DBN.

In yet another embodiment of the present invention the compound of formula (I) prepared by the process of present invention is useful in the preparation of Quetiapine fumarate.

The present invention is exemplified by the following example, which is provided for illustration only and should not be construed to limit the scope of the invention.

### EXAMPLE I

### Step (i) Preparation of 2-(2-nitrophenylthio)benzoic Acid (IV).

To the suspension of potassium carbonate (268 g ) in dimethylformamide (500 ml) thiosalicylic acid (100 g) was added and heated to 80° C. To the reaction mixture 2-fluoronitrobenzene (91.5 g) was added at 80-85° C. Reaction mixture was stirred at 100-110° C. till completion of reaction. After completion of reaction the reaction mixture was cooled and filtered the residue. Filtrate was concentrated to get residue. To the residue, water (200 ml) was added and pH of the solution was adjusted to 11 using sodium hydroxide solution. The aqueous layer was washed with toluene (200 ml). The pH of aqueous layer again adjusted to 2 using conc. hydrochloric acid at 25-30° C. The product obtained was filtered, washed with water (200 ml) and dried under vacuum at 60° C. to get 168-175 gm of titled product. (HPLC purity: 99-100%).

Step (ii & iii) Preparation of 2-nitro-2′-[4-[2-(2-hydroxyethoxy)ethyl]-piperazinylcarbonyl]diphenyl sulphide (VI):

To 2-(2-nitrophenylthio)benzoic acid (100 g) in dichloromethane (850 ml), triethylamine (46 g) and N-methyl morpholine (3.3 g) were added at room temperature and stirred for 15 min. To reaction mixture, solution of pivaloyl chloride (48 g) in dichloromethane (50 ml) was added at 30-35° C. over 30 min. followed by addition of 1-[2-(2-hydroxyethoxy)ethyl]piperazine (61.66 g) and stirred till completion of reaction. The reaction mixture was concentrated to get residue. To the residue, water (300 ml) was added and pH of the solution was adjusted to 2 using conc. hydrochloric acid. The aqueous layer was washed with toluene. The pH of aqueous layer again adjusted to 8.5 using sodium hydroxide solution at 25-30° C. The product was extracted with dichloromethane (2×300 ml). Dichloromethane was distilled out to get 125-130 gm of viscous liquid titled product. (HPLC purity: 97-99%).

Step (iv) Preparation of 2-nitro-2′-[4-[2-((2-acetyloxy)ethoxy)ethyl]-piperazinyl carbonyl]diphenyl sulphide (VII):

To 2-nitro-2′-[4-[2-(2-hydroxyethoxy)ethyl]-piperazinylcarbonyl]diphenylsulphide (130 g), acetic anhydride (318 g) and pyridine (5.9 g) were added and stirred at 28-32° C. till completion of reaction. Acetic anhydride is distilled out under vacuum to get residue. To residue water (400 ml) was added and the product was extracted with dichloromethane (600 ml). The dichloromethane layer was distilled to get 129-131 gm of viscous liquid of titled product. (HPLC purity: 97-99%).

¹H NMR (400 MHz, CDCl₃) δ:2.068 (s, 3H), 2.52-2.54 (m, 2H), 2.62-2.65 (m, 2H), 3.18-3.37 (m, 2H), 3.60-3.87 (m, 8H), 4.20-4.23 (m, 2H), 6.94-6.96 (d, 1H), 7.22-7.27 (m, 1H), 7.36-7.42 (m, 2H), 7.47-7.48 (m, 1H), 7.49-7.60 (m, 2H), 8.18-8.20 (d, 1H).

ESI C₂₃H₂₇N₃SO₆ ( M+1)⁺=474.

Step (v) Preparation of 2-amino-2′-[4-[2-((2-acetyloxy)ethoxy)ethyl]-piperazinylcarbonyl]diphenyl sulphide (VIII):

To 2-nitro-2′-[4-[2-((2-acetyloxy)ethoxy)ethyl]-piperazinylcarbonyl]diphenyl sulphide (130 g) in methanol (1040 ml), 10% palladium on carbon (13 g) was added and the solution was hydrogenated at 50° C. with 10 Kg of hydrogen pressure till completion of reaction. After completion of reaction, reaction mixture was filtered. Methanol was distilled out to get 100-105 gm of viscous liquid of titled product.

HPLC purity: 97-99%.

¹H NMR (400 MHz, CDCl₃) δ:2.07 (s, 3H), 2.39-2.43 (m, 2H), 2.63-2.66 (m, 4H), 3.32-3.34 (m, 2H), 3.62-3.67 (m, 4H), 3.92 (m, 2H), 4.20-4.23 (m, 2H), 4.43-4.45 (bs, 2H), 6.71-6.74 (m, 2H), 7.0-7.07 (m, 1H), 7.16-7.27 (m, 4H), 7.43-7.45 (m, 1H).

ESI C₂₃H₂₉N₃SO₄ (M+1)⁺=444.

Step (vi) Preparation of 11-[4-[2-(2-(2-acetyloxy)ethoxy)ethyl]-1-piperazinvl]dibenzo [b,f][1,4]thiazepine:

To 2-amino-2′-[4-[2-((2-acetyloxy)ethoxy)ethyl]-piperazinylcarbonyl]diphenylsulphide (25 g) in toluene (25 ml) phosphorus oxychloride (25 ml) was added and the resulting mass was stirred at reflux temperature for 5-6 hours. The excess phosphorus oxychloride and toluene were distilled out under vacuum to get thick mass. To the reaction mass, water was added and pH of the reaction mixture was adjusted to 8 using sodium hydroxide solution. The product was extracted with toluene (180 ml). Toluene layer was subjected to carbon treatment, and then concentrated under vacuum to get 11-[4-[2-(2-(2-acetyloxy)ethoxy)ethyl]-1-piperazinyl]dibenzo [b,f][1,4] thiazepine.

¹H NMR (400 MHz, CDCl₃) δ:2.04 (s, 3H), 2.54-2.67 (m, 6H), 3.64-3.68 (m, 8H), 4.20-4.23 (m, 2H), 6.87-6.88 (m, 1H), 7.07-7.08 (m, 1H), 7.16-7.17 (m, 1H), 7.30-7.32 (m, 3H), 7.37-7.39 (m, 1H), 7.49-7.51 (m, 1H).

ESI C₂₃H₂₇N₃SO₃ (M+1)⁺=426.

Step (vii) Preparation of 11-[4-[2-(2-hydroxyethoxy)ethvl]-1-piperazinyl]dibenzo[b,f][1,4]thiazepine (guetiapine) (I):

To 11-[4-[2-(2-(2-acetyloxy)ethoxy)ethyl]-1-piperazinyl]dibenzo [b,f][1,4]thiazepine in methanol sodium hydroxide solution (4g in 10 ml) was added and heated to 160° C. till completion of reaction. Excess water was added and product was extracted with toluene layer. Toluene layer was concentrated under vacuum to get 18-20 gm of quetiapine

### EXAMPLE II

### Step (i) Preparation of 2-(2-nitrophenylthio)benzoic acid (IV).

To the suspension of potassium carbonate (134.3 g) in dimethylformamide (250 ml), thiosalicylic acid (50 g) was added and heated to 80° C. To the reaction mixture 2-chloro nitrobenzene (51.1 g) was added at 80-85° C. Reaction mixture was stirred at 100-110° C. till completion of reaction. After completion of reaction, the reaction mixture was cooled and filtered the residue. Filtrate was concentrated to get residue. To the residue, water (100 ml) was added and pH of the solution was adjusted to 11 using sodium hydroxide solution. The aqueous layer was washed with toluene (100 ml). The pH of aqueous layer again adjusted to 2 using conc. hydrochloric acid at 25-30° C. The product obtained was filtered, washed with water (200 ml) and dried under vacuum at 60° C. to get 82-86 gm of titled product. (HPLC purity: 99-100%).

Steps ii, iii, iv and v are prepared as given in Example-1

Step (vi & vii) Preparation of 11-[4-[2-(2-hydroxyethoxy)ethyl]-1-pinerazinyl]dibenzo[b,f][1,4]thiazepine (quetiapine) (I)

To 2-amino-2′-[4-[2-((2-acetyloxy)ethoxy)ethyl]-piperazinylcarbonyl]diphenyisulphide (25 g) in toluene (25 ml) phosphorus oxychloride (25 ml) was added and the resulting mass was stirred at reflux for 5-6 hours. The excess phosphorus oxychloride and toluene were distilled out under vacuum to get thick mass. To reaction mass water was added and pH of the reaction mixture was adjusted to 12 using sodium hydroxide solution. The reaction mixture was heated at 50° C. till completion of reaction. After completion of reaction, the reaction mixture was cooled to RT and the product was extracted with toluene (180 ml). Toluene layer was subjected to carbon treatment, and then concentrated under vacuum to get 18-20 gm of quetiapine.

### EXAMPLE III

### Preparation of Quetiapine Fumarate:

To quetiapine (5.2 g) prepared according to example (I or II), in isopropyl alcohol (40 ml), fumaric acid (1.26 g) was added and stirred under reflux for 1 hour. The solution was cooled to 30° C. and filtered and dried under vacuum at 60° C. to get 4.8 g, quetiapine fumarate as white solid. Melting point: 172-174° C.

1. A process for the preparation of compound of formula (I) 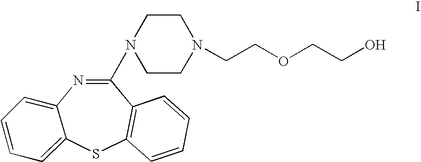
the said process comprising the steps of:
(i) reacting the compound of formula (II) 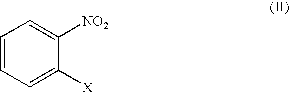
wherein X represents halogen atoms selected from chlorine, bromine, fluorine and iodine with compound of formula (III) 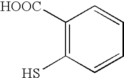
in presence of base and a solvent, to produce acid of formula (IV), 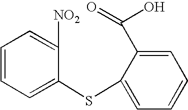
(ii) converting the acid of formula (IV) into acid derivative of formula (V) 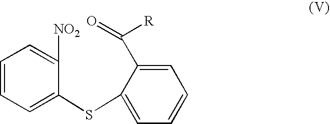
wherein R is a group which forms a basis that a compound of formula (V) in a reactive form including halogen atoms selected from chlorine ,bromine ; a group which forms together with the —C═O group to which R is attached an active ester and a group which forms together with the —C═O group to which Y is attached a mixed anhydride in the presence of a base and solvent to produce the compound of formula (V),
(iii) reacting the compound of formula (V) with compound of formula (X) 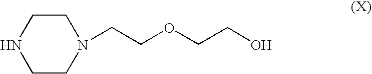
in the presence of base and solvent, to produce compound of formula (VI), 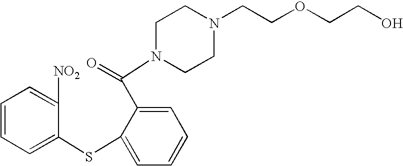
(iv) converting the compound of formula (VI) into compound of formula (VII), 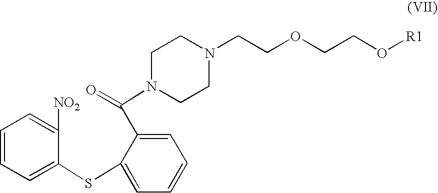
wherein R₁ represents an alcohol protecting group, using a reagent in the presence of base and solvent,
(v) reducing the compound of formula (VII) using a reducing agent in the presence of a solvent to produce compound of formula (VIII),
(vi) cyclizing the compound of formula (VIII) 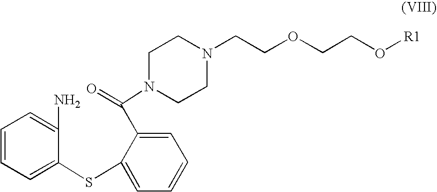
using cyclising agent in the presence of a solvent to produce compound of formula (IX), and 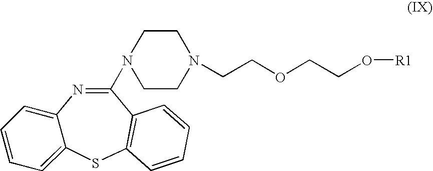
(vii) deprotecting the compound of formula (IX) using basic reagent in the presence of solvent to produce compound of formula (I).

2. A process according to claim 1, wherein X represents halogen atom such as fluoro, chloro, bromo and iodo.

3. A process according to claim 1, wherein the solvent employed in step (i) is selected from acetone, methyl isobutyl ketone, ethyl methyl ketone; hydrocarbon such as benzene, toluene, DMF, DMAc, and the like or mixtures thereof.

4. A process according to claim 1, wherein the base employed in step (i) is selected from alkali/alkaline earth metal hydroxide sodium hydroxide, potassium hydroxide; alkali/alkaline earth metal carbonates such as sodium carbonate, potassium carbonate; alkali/alkaline earth metal bicarbonates such as potassium bicarbonate, metal alkoxide such as sodium methoxide, potassium tert-butoxide; metal amides, metal hydrides such as sodium hydride; organic amines such as triethyl amines , N-methyl-morpholine, diethyl amine, pyridine, DBU, or DBN.

5. A process according to claim 1, wherein the reagent employed in step (ii) is selected from SOCl₂, PCl₅, PCl₃, POCl₃, pivaloyl chloride, ethyl chloroformate, 2,4,6-trichlorobenzoyl chloride, N,N′-carbonyldiimidazole, diethylchlorophophite, diphenylphosphorochloridate, 1-hydroxybenzotriazole or mixtures thereof.

6. A process according to claim 1, wherein the organic solvent employed in step (ii) is selected from halogenated hydrocarbon such as dichloro methane, dichloro ethane; DMF, DMAc, and the like or mixtures thereof.

7. A process according to claim 1, wherein base employed in step (ii) is selected from organic amines such as triethyl amine, N-methyl-morpholine, diethyl amine, pyridine or mixtures thereof.

8. A process according to claim 1, wherein the alcohol-protecting group represented by R₁ is selected from acyl, alkyl, trityl, or benzyl.

9. A process according to claim 1, wherein the base employed in step (iv) is selected from triethyl amine, N-methyl-morpholine, diethyl amine, or pyridine

10. A process according to claim 1, wherein the reagent employed in step (iv) is selected from acetic anhydride, alkyl halide, dialkyl sulphate, trityl chloride, or benzyl halide.

11. A process according to claim 1, wherein the reducing agent use in step (v) selected from Zn, Sn or Fe/acid, AlH₃—AlCl₃, hydrazine, hydrazine/Raney nickel, sulfides such as NaHS, (NH₄)₂S, NaBH₂S₃, or by catalytic hydrogenation using Pd-C, Pt, or Raney nickel

12. A process according to claim 1, wherein the solvent employed in step (v) is selected from esters such as ethyl acetate, methyl acetate; alkanols such as methanol, ethanol, IPA; and the like or mixtures thereof.

13. A process according to claim 1, wherein the acidic reagent used for cyclization in step (vi) is selected from polyphophosporic acid, methane sulphonic acid, PTSA, H₂SO₄, P₂O₅, P₂O₃, PCl₅, PCl₃, PBr₃, PBr₅, SOCl₂, superacids, anhydrides of superacids, Lewis acid, acetic acid, SO₂Cl₂, POCl₃, POBr₃, SOBr₂, formic acid, acetic anhydride and the like.

14. A process according to claim 1, wherein the solvent employed in step (vi) is selected from esters such as ethyl acetate, methyl acetate; alkanols such as methanol, ethanol, IPA; nitrile such as acetonitrile; ketones such as acetone, ethyl methyl ketone; hydrocarbon such as benzene, toluene halogenated hydrocarbon such as dichloro methane, dichloro ethane; DMF, DMAc, diglyme, monoglyme, THF, dioxane, dimethylsulphoxide (DMSO), sulpholane, water and the like or mixtures thereof.

15. A process according to claim 1, wherein the solvent employed in step (vii) is selected from esters such as ethyl acetate, methyl acetate; alkanols such as methanol, ethanol, IPA; water and the like or mixtures thereof.

16. A process according to claim 1 wherein the basic reagent employed in step (vii) is selected from alkali/alkaline earth metal hydroxide such as sodium hydroxide, alkali/alkaline earth metal carbonates such as potassium carbonate, alkali/alkaline earth metal bicarbonates sodium bicarbonate, metal alkoxide such as sodium methoxide, potassium tert-butoxide, metal amides, metal hydrides, organic amines, DBU, or DBN.

17. A compound of formula (VI)

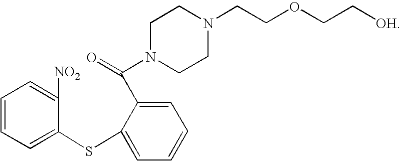

18. A compound of formula (VII), 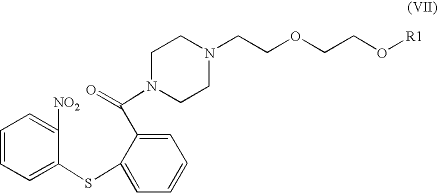
where R₁ is alcohol protecting group such as acyl, alkyl, trityl, or benzyl.

19. A compound of formula (VIII), 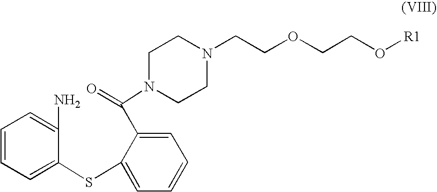
where R₁ is alcohol protecting group such as acyl, alkyl, trityl, or benzyl.

20. A compound of formula (IX), 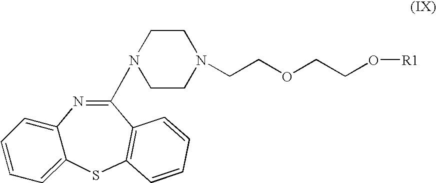
where R₁ is alcohol protecting group such as acyl, alkyl, trityl, or benzyl.
